SEQUENCE LISTING (1 sequence)

SEQ ID NO: 1 - protein, 302 aa, Homo sapiens
MCAERLGQFMTLALVLATFDPARGTDATNPPEGPQDRSSQQKGRLSLQNTAEIQHCLVNAGDVGCGVFECFENNSCEIRGLHGICMTFLHNAGKFDAQGKSFIKDALKCKAHALRHRFGCISRKCPAIREMVSQLQRECYLKHDLCAAAQENTRVIVEMIHFKDLLLHEPYVDLVNLLLTCGEEVKEAITHSVQVQCEQNWGSLCSILSFCTSAIQKPPTAPPERQPQVDRTKLSRAHHGEAGHHLPEPSSRETGRGAKGERGSKSHPNAHARGRVGGLGAQGPSGSSEWEDEQSEYSDIRR

(19) United States
(12) Patent Application Publication
(10) Pub. No.: US 2025/0099547 A1
(43) Pub. Date: Mar. 27, 2025

(54) METHODS FOR TREATING STROKE USING A STANNIOCALCIN 2 (STC2) PHARMACEUTICAL COMPOSITION

(71) Applicant: The Board of Trustees of the Leland Stanford Junior University, Stanford, CA (US)

(72) Inventors: Paul George, Stanford, CA (US); Sruthi Santhanam, Stanford, CA (US); Matine Azadian, Redwood City, CA (US); Byeongtaek Oh, Redwood City, CA (US)

(21) Appl. No.: 18/833,818

(22) PCT Filed: Feb. 6, 2023
(86) PCT No.: PCT/US2023/062035

§ 371 Date: Jul. 26, 2024

Related U.S. Application Data
(60) Provisional application No. 63/307,835, filed on Feb. 8, 2022.

(51) Int. Cl. A61K 38/22; A61K 9/00; A61P 25/28

(52) U.S. Cl. CPC A61K 38/22; A61K 9/0019; A61K 9/0085; A61P 25/28

(57) ABSTRACT

Compositions and methods are provided for enhancing restoration of functional deficits following ischemia-induced damage to the brain. It is shown herein the administration of a stanniocalcin 2 (STC2) agent after an incident of ischemia-induced damage to the brain is beneficial in the restoration of function.

### CROSS REFERENCE TO OTHER APPLICATIONS

This application claims the benefit of U.S. Provisional Application No. 63/307,835, filed Feb. 8, 2022, the contents of which are hereby incorporated by reference in its entirety.

### STATEMENT REGARDING FEDERALLY SPONSORED RESEARCH

This invention was made with Government support under contract NS089976 awarded by the National Institutes of Health. The Government has certain rights in the invention.

### INCORPORATION BY REFERENCE LISTING PROVIDED AS A TEXT

A sequence listing is provided herewith as a sequence listing xml, “S22-005_STAN-1937WO_SeqListing” created on Feb. 6, 2023, and having a size of 2,067 Bytes. The contents of the sequence listing xml are incorporated by reference herein in their entirety.

### BACKGROUND

Of the over 10 million strokes that occur annually, the majority of survivors are left with functional deficits. Early in development the brain is quite plastic, but as the human brain ages, its intrinsic capabilities to recover from insults such as stroke decline. Exogenous cell therapeutics, including neural progenitor cell (NPC) delivery, have emerged in an attempt to offset stroke-induced neuronal loss and promote functional recovery. Alternatively, augmentation of the brain's intrinsic mechanisms of repair such as endogenous stem cell production has been utilized to enhance restoration following ischemia.

Stroke can cause temporary or permanent disabilities, creating an unmet need for therapeutic intervention, which is addressed by the present disclosure.

### SUMMARY

Compositions and methods are provided for enhancing restoration of functional deficits following ischemia-induced damage to the brain. It is shown herein the administration of a stanniocalcin 2 (STC2) agent after an incident of ischemia-induced damage to the brain is beneficial in the restoration of function.

Without being limited to the theory, it is believed that STC2 stimulates endogenous neural progenitor cells to regenerate damaged tissue. The data presented herein demonstrate that administration of STC2 protein, e.g. administration into the brain or CSF, leads to an increase in migratory neuroblasts toward the peri-infarct region and an improvement in behavioral outcome in a stroke model.

In one aspect, the present invention provides a method of enhancing restoration of function following ischemia-induced damage to the brain in a living subject. The method includes the step of administering to a subject in need thereof an effective dose of an STC2 agent. In some embodiments the STC2 agent is an STC2 polypeptide, e.g. human STC2 protein. The protein may be reference (wild-type) protein, or a variant thereof. A variant may be modified to enhance stability in vivo. In some embodiments the STC2 agent is a polynucleotide encoding an STC2 polypeptide. In some embodiments the STC2 agent is delivered by intra-CSF administration, i.e. by direct administration into the CSF.

An individual subject may be selected for treatment following assessment that the individual has suffered ischemia-induced damage to the brain, which assessment can be performed by functional assessment, imaging, and the like, for example by EEG (brain nerve cell activity), CAT scan to assess brain damage and MRI to obtain internal body visuals.

Administration of the STC2 agent is performed following ischemic damage, e.g. after about 2 hours, 6 hrs, 12 hours, 24 hours, or more. In some embodiments administration is performed one or more days following the ischemic damage, e.g. after about 1 day, 2 days, 3 days, 4 days, 5 days, 6, days, 7 days, or more. In some embodiments, the administration is performed from about 1 to 7 days, from about 2 to 7 days, from about 3 to 7 days, following ischemic damage, and may be continued for a period of time sufficient for restoration of function, e.g. for about 1 week, 2 weeks, 3 weeks, 4 weeks, 2 months, 3 months or more.

In some embodiments an individual with ischemia-induced damage to the brain is characterized with stroke, which can be categorized as ischemic, hemorrhagic, or subarachnoid.

In some embodiments, compositions are provided for restoration of functional deficits following ischemia-induced damage to the brain, comprising a therapeutically effective amount of an STC2 agent and a pharmaceutically acceptable carrier. Methods are also provided for manufacturing a medicament for use in restoration of functional deficits following ischemia-induced damage to the brain in living subjects in need thereof, comprising providing a therapeutically effective amount of an STC2 agent in a pharmaceutical carrier. Methods are also provided for manufacturing medicaments for use in restoration of functional deficits following ischemia-induced damage to the brain for treatment of each of the conditions, diseases and disorders described herein.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1. Intraventricular delivery of recombinant STC2 protein in rodents. Schematic of the brain slice view (left) and top view (right) in the skull for STC2 protein delivery via mini-osmotic pump. The cannula of the osmotic pump is implanted at 0.8 mm posterior (Y) and 1.5 mm contralateral (X) to the bregma at a depth of 3.5 mm at one-week after stroke.

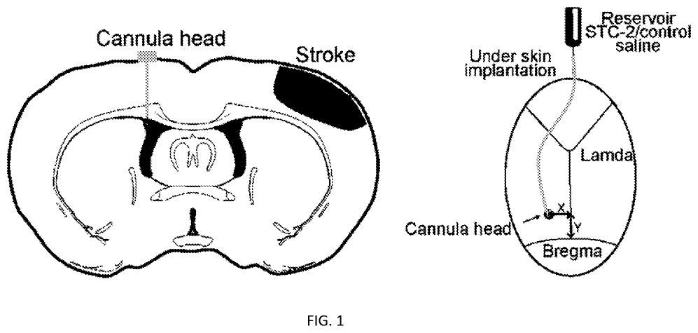

FIG. 2. Intraventricular STC-2 improves functional recovery. Our results indicate that intraventricular injection of STC-2 alone post-stroke improves functional recovery in comparison to a control and intraventricular saline group. Functional recovery was assessed by vibrissae-forepaw test post-stroke (n=10 per group, analyzed using log-two-way ANOVA, followed by Tukey's HSD post hoc test with *P<0.05, and **P<0.01).

FIG. 3. Intraventricular STC2 increases endogenous stem cell proliferation. Histological analysis reveals that intraventricular administration of STC2 protein leads to an increase in endogenous neuroblasts migrating toward the peri-infarct region. a.) Representative images of DCX+/BrdU+ cells in subventricular zone. b.) Representative images of DCX+/BrdU+ cells in peri-infarct region. c.) Analyzed using one-way ANOVA, followed by Tukey's HSD post hoc test with *P<0.05. Data shown as mean±SEM, n=20 images/4 rats per group.

FIG. 4. Stability of recombinant STC-2 in-vitro. The duration at which STC-2 would remain detectable with a standard STC-2 ELISA assay under normal physiological conditions (static artificial CSF at 37 degrees celsius) was determined.

FIG. 5. Stability of recombinant STC-2 in-vivo. STC-2 (10 mg/mL) was injected into the circulating CSF space (via cisterna magna) of rodents (n=3), and the CSF was sampled at each timepoint thereafter up to 12 hrs. We assessed the rough half-life of CSF and the point at which it was no longer detectable (at 10 h post-injection) after a single bolus injection with a standard STC-2 Elisa kit.

FIG. 6. Neuroprotective potential/dose needed for STC-2 to exhibit an effect on the viability of iPSC derived human neuronal cells (in culture) after OGD (oxygen and glucose deprivation. The results indicate that when 4 ng/ml of STC-2 was given prior to 3 hrs of OGD, it conferred a significant neuroprotective effect on the neuronal cells (p<0.05).

### DETAILED DESCRIPTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

By “ischemic episode” is meant any circumstance that results in a deficient supply of blood to a tissue. When the ischemia is associated with a stroke, it can be either global or focal ischemia, as defined below. The term “ischemic stroke” refers more specifically to a type of stroke that is of limited extent and caused due to blockage of blood flow. Cerebral ischemic episodes result from a deficiency in the blood supply to the brain. The spinal cord, which is also a part of the central nervous system, is equally susceptible to ischemia resulting from diminished blood flow.

The term “ischemic damage” refers to loss of function following an ischemic episode, and can include, for example, the death of neurons in selectively vulnerable regions throughout the brain.

By “focal ischemia,” as used herein in reference to the central nervous system, is meant the condition that results from the blockage of a single artery that supplies blood to the brain or spinal cord, resulting in damage to the cells in the territory supplied by that artery.

By “global ischemia,” as used herein in reference to the central nervous system, is meant the condition that results from a general diminution of blood flow to the entire brain, forebrain, or spinal cord, which causes the death of neurons in selectively vulnerable regions throughout these tissues. The pathology in each of these cases is quite different, as are the clinical correlates. Models of focal ischemia apply to patients with focal cerebral infarction, while models of global ischemia are analogous to cardiac arrest, and other causes of systemic hypotension.

The term “stroke” broadly refers to the development of neurological deficits associated with impaired blood flow to the brain regardless of cause. Potential causes include, but are not limited to, thrombosis, hemorrhage and embolism. Current methods for diagnosing stroke include symptom evaluation, medical history, EEG (brain nerve cell activity), CAT scan to assess brain damage and MRI to obtain internal body visuals. Thrombus, embolus, and systemic hypotension are among the most common causes of cerebral ischemic episodes. Other injuries may be caused by hypertension, hypertensive cerebral vascular disease, rupture of an aneurysm, an angioma, blood dyscrasias, cardiac failure, cardiac arrest, cardiogenic shock, septic shock, head trauma, spinal cord trauma, seizure, bleeding from a tumor, or other blood loss.

Stroke can be categorized as ischemic, hemorrhagic, or subarachnoid. Ischemic strokes present acutely, and establishing the time of symptom onset may be critical. If the time of symptom onset is unknown, the time the patient was last known to be normal without new neurological symptoms is used.

A neurological exam is generally performed for all patients suspected of stroke. The National Institutes of Health Stroke Scale (NIHSS) is commonly used to measure the severity of the stroke and has 11 categories and a score that ranges from 0 to 42. The 11 categories include the level of consciousness (LOC), which incorporates LOC questions evaluating best gaze, visual, facial palsy, motor arm, motor leg, limb ataxia, sensory, best language, dysarthria, and extinction and inattention. The stroke scale should be performed in the order listed.

A plain CT head or brain MRI is recommended for patients within 20 minutes of presentation to rule out hemorrhage. In hospitals that are stroke centers or can provide emergency care, vascular imaging should be considered for possible endovascular intervention; however, this should not delay the administration of thrombolytics. Other diagnostic tests include an electrocardiogram (ECG), troponin, complete blood count, electrolytes, blood urea nitrogen (BUN), creatinine (Cr), and coagulation factors. A complete blood count can look for anemia or suggest infection. Electrolyte abnormalities should be corrected. BUN and Cr should be monitored as contrast studies may worsen kidney function. Coagulation factors, including PTT, PT, and INR, should also be done as the elevated levels can suggest a cause of hemorrhagic stroke.

Initial treatment in acute ischemic stroke is to preserve tissue in areas where perfusion is decreased but sufficient to avoid infarction. Tissue in this area of oligemia is preserved by restoring blood flow to the compromised regions and improving collateral flow. Recanalization strategies include recombinant tissue-type plasminogen activator. Restoring blood flow can minimize the effects of ischemia only if performed quickly. The AHA/ASA recommends intravenous (IV) alteplase for patients who satisfy inclusion criteria and have symptom onset or last known baseline within 3 hours. Inclusion criteria include diagnosis of ischemic stroke with “measurable neurological deficit,” symptom onset within 3 hours before treatment, and age 18 years or older.

The middle cerebral artery (MCA) is the most common artery involved in stroke. It supplies a large area of the lateral surface of the brain and part of the basal ganglia and the internal capsule via four segments (M1, M2, M3, and M4). The M1 (horizontal) segment supplies the basal ganglia, which is involved in motor control, motor learning, executive function, and emotions. The M2 (Sylvian) segment supplies the insula, superior temporal lobe, parietal lobe, and the inferolateral frontal lobe.

The anterior cerebral artery (ACA) provides blood supply to the frontal, prefrontal, primary motor, primary sensory, and supplemental motor cortices. Pure ACA infarcts are uncommon because of the significant collateral blood supply provided by the anterior circulating artery. The sensory and motor cortices receive sensory information and control movement of the contralateral lower extremity. The supplemental motor area contains the Broca area, which is involved in the initiation of speech. The prefrontal cortex is used to organize and plan complex behavior and is thought to influence the personality.

The superficial posterior cerebral artery (PCA) supplies the occipital lobe and the inferior portion of the temporal lobe, while the deep PCA supplies the thalamus and the posterior limb of the internal capsule, as well as other deep structures of the brain. The occipital lobe is the location of the primary and secondary visual areas, where sensory input from the eyes is interpreted. The thalamus relays information between the ascending and descending neurons, while the internal capsule contains the descending fibers of the lateral and ventral corticospinal tracts.

Stanniocalcin 2 (STC2) is a glycosylated, disulfide-linked, homodimeric hormone. Human STC2 protein is 302 amino acids in length, with first 24 residues predicted to be a signal peptide and remaining residues comprise the mature form of the hormone. There are a number of conserved cysteine residues and N-linked glycosylation consensus sequence. STC2 is phosphorylated by casein kinase 2 on its serine residues. The C-terminal of STC2 has a cluster of histidine residues. In is expressed in a variety of tissues including pancreas, heart, placenta, spleen, lung, kidneys, skeletal muscles, brain, lungs, liver, and kidneys. It has been indicated that STC2 may be secreted. The reference sequence for human STC2 mRNA may be accessed at Genbank NM_003714. The reference sequence for human STC2 protein may be accessed at Genbank NP_003705. The protein may be produced by recombinant methods as known in the art.

For example, the human reference protein may have a sequence as set forth in SEQ ID NO: 1: MCAERLGQFM TLALVLATFD PARGTDATNP PEGPQDRSSQ QKGRLSLQNT AEIQHCLVNA GDVGCGVFEC FENNSCEIRG LHGICMTFLH NAGKFDAQGK SFIKDALKCK AHALRHRFGC ISRKCPAIRE MVSQLQRECY LKHDLCAAAQ ENTRVIVEMI HFKDLLLHEP YVDLVNLLLT CGEEVKEAIT HSVQVQCEQN WGSLCSILSF CTSAIQKPPT APPERQPQVD RTKLSRAHHG EAGHHLPEPS SRETGRGAKG ERGSKSHPNA HARGRVGGLG AQGPSGSSEW EDEQSEYSDI RR; and in some embodiments may have a sequence of the mature form of SEQ ID NO:1, e.g. residues 25-302.

In some embodiments an STC2 protein can be conjugated to additional molecules to provide desired pharmacological properties, such as extended half-life. In one embodiment, STC2 protein can be fused to the Fc domain of IgG, albumin, or other molecules to extend its half-life, e.g. by pegylation, glycosylation, and the like as known in the art. In some embodiments the STC2 protein is conjugated to a polyethylene glycol molecules or “PEGylated.” The PEG conjugated to the polypeptide sequence may be linear or branched. The PEG may be attached directly to the polypeptide or via a linker molecule. The processes and chemical reactions necessary to achieve PEGylation of biological compounds is well known in the art.

An STC2 protein can be acetylated at the N-terminus, using methods known in the art, e.g. by enzymatic reaction with N-terminal acetyltransferase and, for example, acetyl CoA. STC2 protein can be acetylated at one or more lysine residues, e.g. by enzymatic reaction with a lysine acetyltransferase. See, for example Choudhary et al. (2009). Science. 325 (5942): 834-840.

Fc-fusion can also endow alternative Fc receptor mediated properties in vivo. The “Fc region” can be a naturally occurring or synthetic polypeptide that is homologous to an IgG C-terminal domain produced by digestion of IgG with papain. IgG Fc has a molecular weight of approximately 50 kDa. The polypeptides can include the entire Fc region, or a smaller portion that retains the ability to extend the circulating half-life of a chimeric polypeptide of which it is a part. In addition, full-length or fragmented Fc regions can be variants of the wild-type molecule. That is, they can contain mutations that may or may not affect the function of the polypeptides, where native activity is not necessary or desired in all cases.

In other embodiments, an STC2 protein can comprise a polypeptide sequence that functions as an antigenic tag, such as a FLAG sequence. FLAG sequences are recognized by biotinylated, highly specific, anti-FLAG antibodies, as described herein (see also Blanar et al., Science 256:1014, 1992; LeClair et al., Proc. Natl. Acad. Sci. USA 89:8145, 1992). In some embodiments, the chimeric polypeptide further comprises a C-terminal c-myc epitope tag.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

The term “sequence identity,” as used herein in reference to polypeptide or DNA sequences, refers to the subunit sequence identity between two molecules. When a subunit position in both of the molecules is occupied by the same monomeric subunit (e.g., the same amino acid residue or nucleotide), then the molecules are identical at that position. The similarity between two amino acid or two nucleotide sequences is a direct function of the number of identical positions. In general, the sequences are aligned so that the highest order match is obtained. If necessary, identity can be calculated using published techniques and widely available computer programs, such as the GCS program package (Devereux et al., Nucleic Acids Res. 12:387, 1984), BLASTP, BLASTN, FASTA (Atschul et al., J. Molecular Biol. 215:403, 1990).

By “protein variant” or “variant protein” or “variant polypeptide” herein is meant a protein that differs from a wild-type protein by virtue of at least one amino acid modification. The parent polypeptide may be a naturally occurring or wild-type (WT) polypeptide, or may be a modified version of a WT polypeptide. Variant polypeptide may refer to the polypeptide itself, a composition comprising the polypeptide, or the amino sequence that encodes it. The variant polypeptide may have at least one amino acid modification compared to the parent polypeptide, e.g. from about one to about ten amino acid modifications, and may have from about one to about five amino acid modifications compared to the parent.

In some embodiments an STC2 agent has at least 75% sequence identity to a reference (wild type) human STC2 sequence, at least 80% sequence identity, at least 85%, at least 90%, at least 95%, at least 99% sequence identity. Some agents contain a functional fragment of STC2, comprising at least a portion of the reference sequence Genbank NP_003705, e.g. comprising at least 5 contiguous amino acids, at least 10 contiguous amino acids, at least 20 amino acids, at least 30 amino acids, at least 40 amino acids, at least 50 amino acids, which may be provided as a fusion protein joined to, e.g. a domain that provides for enhanced half-life and stability after administration.

By “parent polypeptide”, “parent protein”, “precursor polypeptide”, or “precursor protein” as used herein is meant an unmodified polypeptide that is subsequently modified to generate a variant. A parent polypeptide may be a wild-type (or native) polypeptide, or a variant or engineered version of a wild-type polypeptide. Parent polypeptide may refer to the polypeptide itself, compositions that comprise the parent polypeptide, or the amino acid sequence that encodes it.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. “Amino acid analogs” refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acid modifications disclosed herein may include amino acid substitutions, deletions and insertions, particularly amino acid substitutions. Variant proteins may also include conservative modifications and substitutions at other positions of the cytokine and/or receptor (e.g., positions other than those involved in the affinity engineering). Such conservative substitutions include those described by Dayhoff in The Atlas of Protein Sequence and Structure 5 (1978), and by Argos in EMBO J., 8:779-785 (1989). For example, amino acids belonging to one of the following groups represent conservative changes: Group I: Ala, Pro, Gly, Gln, Asn, Ser, Thr; Group II: Cys, Ser, Tyr, Thr; Group III: Val, 11e, Leu, Met, Ala, Phe; Group IV: Lys, Arg, His; Group V: Phe, Tyr, Trp, His; and Group VI: Asp, Glu. Further, amino acid substitutions with a designated amino acid may be replaced with a conservative change.

Expression construct: STC2 coding sequences may be introduced on an expression vector for delivery to a cell. The nucleic acid encoding an STC2 protein is inserted into a vector for expression and/or integration. Many such vectors are available. The vector components generally include, but are not limited to, one or more of the following: an origin of replication, one or more marker genes, an enhancer element, a promoter, and a transcription termination sequence. Vectors include viral vectors, plasmid vectors, integrating vectors, and the like.

Expression vectors may contain a selection gene, also termed a selectable marker. This gene encodes a protein necessary for the survival or growth of transformed host cells grown in a selective culture medium or a truncated gene encoding a surface marker that allows for antibody based detection. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, or (d) enable surface antibody based detection for isolation via fluoresences activating cell sorting (FACS) or magnetic separation e.g. truncated forms of NGFR, EGFR, CD19.

Nucleic acids are “operably linked” when placed into a functional relationship with another nucleic acid sequence. For example, DNA for a signal sequence is operably linked to DNA for a polypeptide if it is expressed as a preprotein that signals the secretion of the polypeptide; a promoter or enhancer is operably linked to a coding sequence if it affects the transcription of the sequence; and a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous.

Expression vectors will contain a promoter that is recognized by the host organism and is operably linked to the construct coding sequence. Promoters are untranslated sequences located upstream (5′) to the start codon of a structural gene (generally within about 100 to 1000 bp) that control the transcription and translation of particular nucleic acid sequence to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known.

Transcription from vectors in mammalian host cells may be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus LTR (such as murine stem cell virus), hepatitis-B virus and Simian Virus 40 (SV40), from heterologous mammalian promoters, e.g., the actin promoter, PGK (phosphoglycerate kinase), or an immunoglobulin promoter, or from heat-shock promoters, provided such promoters are compatible with the host cell systems. The early and late promoters of the SV40 virus are conveniently obtained as an SV40 restriction fragment that also contains the SV40 viral origin of replication.

Transcription by higher eukaryotes may be increased by inserting an enhancer sequence into the vector. Enhancers are cis-acting elements of DNA, usually about from 10 to 300 bp in length, which act on a promoter to increase its transcription. Enhancers are relatively orientation and position independent, having been found 5′ and 3′ to the transcription unit, within an intron, as well as within the coding sequence itself. Many enhancer sequences are now known from mammalian genes (globin, elastase, albumin, α-fetoprotein, and insulin). Typically, however, one will use an enhancer from a eukaryotic virus. Examples include the SV40 enhancer on the late side of the replication origin, the cytomegalovirus early promoter enhancer, the polyoma enhancer on the late side of the replication origin, and adenovirus enhancers. The enhancer may be spliced into the expression vector at a position 5′ or 3′ to the coding sequence, but is preferably located at a site 5′ from the promoter.

Expression vectors for use in eukaryotic host cells will also contain sequences necessary for the termination of transcription and for stabilizing the mRNA. Such sequences are commonly available from the 5′ and, occasionally 3′, untranslated regions of eukaryotic or viral DNAs or cDNAs. Construction of suitable vectors containing one or more of the above-listed components employs standard techniques.

Suitable host cells for cloning a construct are the prokaryotic, yeast, or other eukaryotic cells described above. Examples of useful mammalian host cell lines are mouse L cells (L-M [K-], ATCC #CRL-2648), monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); Chinese hamster ovary cells/-DHFR (CHO); mouse Sertoli cells (TM4); monkey kidney cells (CV1 ATCC CCL 70); African green monkey kidney cells (VERO-76, ATCC CRL-1 587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; and a human hepatoma line (Hep G2).

AAV gene therapy. Utilizing a viral vehicle to deliver genetic material into cells allows direct targeting of pathogenic molecules and restoration of function. Because AAV is non-pathogenic and cannot reproduce itself without helper viruses, it has served as a primary vehicle for gene therapy. It is a single-stranded DNA virus that stably and efficiently infects a wide variety of cells in multiple tissues. AAV2, the best-characterized AAV serotype, can efficiently infects certain neurons.

In some embodiments, the vector is a recombinant adeno-associated virus (AAV) vector. AAV vectors are DNA viruses of relatively small size that can integrate, in a stable and site specific manner, into the genome of the cells that they infect. They are able to infect a wide spectrum of cells without inducing any effects on cellular growth, morphology or differentiation, and they do not appear to be involved in human pathologies. The AAV genome has been cloned, sequenced and characterized. It encompasses approximately 4700 bases and contains an inverted terminal repeat (ITR) region of approximately 145 bases at each end, which serves as an origin of replication for the virus. The remainder of the genome is divided into two essential regions that carry the encapsidation functions: the left-hand part of the genome, that contains the rep gene involved in viral replication and expression of the viral genes; and the right-hand part of the genome, that contains the cap gene encoding the capsid proteins of the virus.

The application of AAV as a vector for gene therapy has been rapidly developed in recent years. Wild-type AAV can infect, with a comparatively high titer, dividing or non-dividing cells, or tissues of mammal, including human, and also can integrate into in human cells at specific site (on the long arm of chromosome 19) (Kotin et al, Proc. Natl. Acad. Sci. U.S.A., 1990. 87:2211-2215; Samulski et al, EMBO J., 1991. 10:3941-3950 the disclosures of which are hereby incorporated by reference herein in their entireties). AAV vector without the rep and cap genes loses specificity of site-specific integration, but may still mediate long-term stable expression of exogenous genes. AAV vector exists in cells in two forms, wherein one is episomic outside of the chromosome; another is integrated into the chromosome, with the former as the major form. Moreover, AAV has not been found to be associated with any human disease, nor any change of biological characteristics arising from the integration has been observed. There are sixteen serotypes of AAV reported in literature, respectively named AAV1, AAV2, AAV3, AAV4, AAV5, AAV6, AAV7, AAV8, AAV9, AAV10, AAV11, AAV12, AAV13, AAV14, AAV15, and AAV16, wherein AAV5 is originally isolated from humans (Bantel-Schaal, and H. zur Hausen. Virology, 1984. 134:52-63), while AAV1-4 and AAV6 are all found in the study of adenovirus (Ursula Bantel-Schaal, Hajo Delius and Harald zur Hausen. J. Viral., 1999. 73:939-947).

AAV vectors may be prepared using any convenient methods. Adeno-associated viruses of any serotype are suitable (See, e.g., Blacklow, pp. 165-174 of “Parvoviruses and Human Disease” J. R. Pattison, ed. (1988); Rose, Comprehensive Virology 3:1, 1974; P. Tattersall “The Evolution of Parvovirus Taxonomy” In Parvoviruses (J R Kerr, S F Cotmore. ME Bloom, RMLinden, C RParrish, Eds.) p 5-14, Rudder Arnold, London, U K (2006); and D E Bowles, J E Rabinowitz, R J Samulski “The Genus Dependovirus” (J R Kerr, SF Cotmore. ME Bloom, R M Linden, C R Parrish, Eds.) p 15-23, Rudder Arnold, London, UK (2006), the disclosures of which are hereby incorporated by reference herein in their entireties). Methods for purifying for vectors may be found in, for example, U.S. Pat. Nos. 6,566,118, 6,989,264, and 6,995,006 and W0/1999/011764 titled “Methods for Generating High Titer Helper-free Preparation of Recombinant AAV Vectors”, the disclosures of which are herein incorporated by reference in their entirety. Preparation of hybrid vectors is described in, for example, PCT Application No. PCTIUS2005/027091, the disclosure of which is herein incorporated by reference in its entirety. The use of vectors derived from the AAVs for transferring genes in vitro and in vivo has been described (See e.g., International Patent Application Publication Nos: 91/18088 and WO 93/09239; U.S. Pat. Nos. 4,797,368, 6,596,535, and 5,139,941; and European Patent No: 0488528, all of which are herein incorporated by reference in their entirety). These publications describe various AAV-derived constructs in which the rep and/or cap genes are deleted and replaced by a gene of interest, and the use of these constructs for transferring the gene of interest in vitro (into cultured cells) or in vivo (directly into an organism). The replication defective recombinant AAVs according to the invention can be prepared by co-transfecting a plasmid containing the nucleic acid sequence of interest flanked by two AAV inverted terminal repeat (ITR) regions, and a plasmid carrying the AAV encapsidation genes (rep and cap genes), into a cell line that is infected with a human helper virus (for example an adenovirus). The AAV recombinants that are produced are then purified by standard techniques.

In some embodiments, the vector(s) for use in the methods of the invention are encapsidated into a virus particle (e.g. AAV virus particle including, but not limited to, AAV1, AAV2, AAV3, AAV4, AAV5, AAV6, AAV7, AAV8, AAV9, AAVIO, AAVII, AAV12, AAV13, AAV14, AAV15, and AAV16). Accordingly, the invention includes a recombinant virus particle (recombinant because it contains a recombinant polynucleotide) comprising any of the vectors described herein. Methods of producing such particles are known in the art and are described in U.S. Pat. No. 6,596,535.

A neuron-specific promoter allows precise manipulation of gene expression without affecting other cell types. Aspects of the present invention encompass expression cassettes and/or vectors comprising polynucleotide sequences of interest for expression in targeted cells. Targeted expression is accomplished using a cell-selective or cell-specific promoter. Examples are promoters for somatostatin, parvalbumin, GABAa6, L7, and calbindin. Other cell specific promoters can be promoters for kinases such as PKC, PKA, and CaMKII; promoters for other ligand receptors such as NMDAR1, NNIDAR2B, GluR2; promoters for ion channels including calcium channels, potassium channels, chloride channels, and sodium channels; and promoters for other markers that label classical mature and dividing cell types, such as calretinin, nestin, and beta3-tubulin.

The term “isolated” refers to a molecule that is substantially free of its natural environment. For instance, an isolated protein is substantially free of cellular material or other proteins from the cell or tissue source from which it is derived. The term refers to preparations where the isolated protein is sufficiently pure to be administered as a therapeutic composition, or at least 70% to 80% (w/w) pure, more preferably, at least 80%-90% (w/w) pure, even more preferably, 90-95% pure; and, most preferably, at least 95%, 96%, 97%, 98%, 99%, or 100% (w/w) pure. A “separated” compound refers to a compound that is removed from at least 90% of at least one component of a sample from which the compound was obtained. Any compound described herein can be provided as an isolated or separated compound.

The terms “subject,” “individual,” and “patient” are used interchangeably herein to refer to a mammal being assessed for treatment and/or being treated. In some embodiments, the mammal is a human. The terms “subject,” “individual,” and “patient” encompass, without limitation, individuals having a disease. Subjects may be human, but also include other mammals, particularly those mammals useful as laboratory models for human disease, e.g., mice, rats, etc.

The term “sample” with reference to a patient encompasses blood and other liquid samples of biological origin, solid tissue samples such as a biopsy specimen or tissue cultures or cells derived therefrom and the progeny thereof. The term also encompasses samples that have been manipulated in any way after their procurement, such as by treatment with reagents; washed; or enrichment for certain cell populations, such as diseased cells. The definition also includes samples that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's diseased cell, e.g., a sample comprising polynucleotides and/or polypeptides that is obtained from a patient's diseased cell (e.g., a cell lysate or other cell extract comprising polynucleotides and/or polypeptides); and a sample comprising diseased cells from a patient. A biological sample comprising a diseased cell from a patient can also include non-diseased cells.

The term “diagnosis” is used herein to refer to the identification of a molecular or pathological state, disease or condition in a subject, individual, or patient.

The term “prognosis” is used herein to refer to the prediction of the likelihood of death or disease progression, including recurrence, spread, and drug resistance, in a subject, individual, or patient. The term “prediction” is used herein to refer to the act of foretelling or estimating, based on observation, experience, or scientific reasoning, the likelihood of a subject, individual, or patient experiencing a particular event or clinical outcome. In one example, a physician may attempt to predict the likelihood that a patient will survive.

As used herein, the terms “treatment,” “treating,” and the like, refer to administering an agent, or carrying out a procedure, for the purposes of obtaining an effect on or in a subject, individual, or patient. The effect may be therapeutic in terms of effecting a partial or complete restoration of function. “Treatment,” as used herein, may include treatment of stroke associated functional deficits in a mammal, particularly in a human.

Treating may refer to any indicia of success in the treatment, including any objective or subjective parameter such as abatement; remission; diminishing of symptoms or making the disease condition more tolerable to the patient. The treatment or amelioration of symptoms can be based on objective or subjective parameters; including the results of an examination by a physician.

As used herein, a “therapeutically effective amount” refers to that amount of the therapeutic agent sufficient to treat a disease or disorder. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Dosage and frequency may vary depending on the half-life of the agent in the patient. It will be understood by one of skill in the art that such guidelines will be adjusted for the molecular weight of the active agent, the clearance from the blood, the mode of administration, and other pharmacokinetic parameters. The dosage may also be varied for localized administration, e.g. intranasal, inhalation, etc., or for systemic administration, e.g. i.m., i.p., i.v., oral, and the like.

In some embodiments a flat dose is administered to an adult human of from about 100 ng of an STC2 agent, e.g. human STC2 protein, to about 1 mg of an STC2 agent, and may be from about 100 ng, 250 ng, 500 ng, 750 ng, 1 μg, 5 μg, 10 μg, 25 μg, 50 μg, 100 μg, 250 μg, 500 μg, 750 μg, or more. In some embodiments a unit dose is from about 500 ng to about 100 μg, from about 1 μg, to about 50 μg. In other embodiments a dose is calculated by surface area of weight of the patient, e.g. from about 1 ng/kg weight, from about 10 ng/kg, 50 ng/kg, 100 ng/kg, 1 μg/kg, 5 μg/kg, 10 μg/kg, or more.

An active agent, e.g. an STC2 protein or coding sequence, can be administered by any suitable means, including intra-CSF, oral, parenteral, intranasal, etc. Parenteral infusions include intramuscular, intravenous (bolus or slow drip), intraarterial, intraperitoneal, intrathecal or subcutaneous administration.

Due to the limited permeability of the blood brain barrier, the methods of the disclosure may utilize direct administration of drugs into the brain, by intra-CSF administration, to achieve full therapeutic effect. As used herein, “intra-CSF administration” means direct administration into the CSF, located in the subarachnoid space between the arachnoid and pia mater layers of the meninges surrounding the brain. Intra-CSF administration can be performed via intra-cisterna magna, intraventricular or intrathecal administration. As used herein, “intra-cisterna magna administration” means administration into the cisterna magna, an opening of the subarachnoid space located between the cerebellum and the dorsal surface of the medulla oblongata. As used herein, “intraventricular administration” means administration into the either of both lateral ventricles of the brain as used herein, “intrathecal administration” involves the direct administration into the CSF within the intrathecal space of the spinal column. Intrathecal delivery methods include intracerebroventricular (ICV), intrathecal-lumbar and intracisternal routes. The ICV route enables the administration of drugs into a lateral cerebral ventricle via an implanted device (reservoir and catheter). As used herein, “intraparenchymal administration” means local administration directly into any region of the brain parenchyma. As used herein, “intranasal administration” means administration by way of the nasal structures.

ICV devices allow administration of drugs either directly into the CSF or by interstitial infusion (with convection). In some embodiments the agent is delivered as intra-CNS administration. In some embodiments administration utilizes an implantable device to deliver the agent, for example an Ommaya reservoir. The device may be implanted intraventricularly, for example, with a conventional stereotaxic apparatus. Sustained release administration is also specifically included in the disclosure, by such means as depot injections or erodible implants.

As noted above, an agent can be formulated with an a pharmaceutically acceptable carrier (one or more organic or inorganic ingredients, natural or synthetic, with which a subject agent is combined to facilitate its application). A suitable carrier includes sterile saline although other aqueous and non-aqueous isotonic sterile solutions and sterile suspensions known to be pharmaceutically acceptable are known to those of ordinary skill in the art. An “effective amount” refers to that amount which is capable of ameliorating or delaying progression of the diseased, degenerative or damaged condition. An effective amount can be determined on an individual basis and will be based, in part, on consideration of the symptoms to be treated and results sought. An effective amount can be determined by one of ordinary skill in the art employing such factors and using no more than routine experimentation.

An agent can be administered as a pharmaceutical composition comprising a pharmaceutically acceptable excipient. The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

As used herein, compounds which are “commercially available” may be obtained from commercial sources including but not limited to Acros Organics (Pittsburgh PA), Aldrich Chemical (Milwaukee WI, including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester PA), Crescent Chemical Co. (Hauppauge NY), Eastman Organic Chemicals, Eastman Kodak Company (Rochester NY), Fisher Scientific Co. (Pittsburgh PA), Fisons Chemicals (Leicestershire UK), Frontier Scientific (Logan UT), ICN Biomedicals, Inc. (Costa Mesa CA), Key Organics (Cornwall U.K.), Lancaster Synthesis (Windham NH), Maybridge Chemical Co. Ltd. (Cornwall U.K.), Parish Chemical Co. (Orem UT), Pfaltz & Bauer, Inc. (Waterbury CN), Polyorganix (Houston TX), Pierce Chemical Co. (Rockford IL), Riedel de Haen AG (Hannover, Germany), Spectrum Quality Product, Inc. (New Brunswick, NJ), TCI America (Portland OR), Trans World Chemicals, Inc. (Rockville MD), Wako Chemicals USA, Inc. (Richmond VA), Novabiochem and Argonaut Technology.

Compounds useful for administration with the active agents of the invention can also be made by methods known to one of ordinary skill in the art. As used herein, “methods known to one of ordinary skill in the art” may be identified through various reference books and databases. Suitable reference books and treatises that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C. may be contacted for more details). Chemicals that are known but not commercially available in catalogs may be prepared by custom chemical synthesis houses, where many of the standard chemical supply houses (e.g., those listed above) provide custom synthesis services.

The active agents are incorporated into a variety of formulations for therapeutic administration. In one aspect, the agents are formulated into pharmaceutical compositions by combination with appropriate, pharmaceutically acceptable carriers or diluents, and are formulated into preparations in solid, semi-solid, liquid or gaseous forms, such as tablets, capsules, powders, granules, ointments, solutions, suppositories, injections, inhalants, gels, microspheres, and aerosols. As such, administration of the active agents and/or other compounds can be achieved in various ways, usually by oral administration. The active agents and/or other compounds may be systemic after administration or may be localized by virtue of the formulation, or by the use of an implant that acts to retain the active dose at the site of implantation.

In pharmaceutical dosage forms, the active agents may be administered in the form of their pharmaceutically acceptable salts, or they may also be used alone or in appropriate association, as well as in combination with other pharmaceutically active compounds. The agents may be combined, as previously described, to provide a cocktail of activities. The following methods and excipients are exemplary and are not to be construed as limiting the invention.

Formulations are typically provided in a unit dosage form, where the term “unit dosage form,” refers to physically discrete units suitable as unitary dosages for human subjects, each unit containing a predetermined quantity of active agent in an amount calculated sufficient to produce the desired effect in association with a pharmaceutically acceptable diluent, carrier or vehicle. The specifications for the unit dosage forms of the present invention depend on the particular complex employed and the effect to be achieved, and the pharmacodynamics associated with each complex in the host.

The pharmaceutically acceptable excipients, such as vehicles, adjuvants, carriers or diluents, are commercially available. Moreover, pharmaceutically acceptable auxiliary substances, such as pH adjusting and buffering agents, tonicity adjusting agents, stabilizers, wetting agents and the like, are commercially available. Any compound useful in the methods and compositions of the invention can be provided as a pharmaceutically acceptable base addition salt. “Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

In some embodiments, pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes).

A carrier may bear the agents in a variety of ways, including covalent bonding either directly or via a linker group, and non-covalent associations. Suitable covalent-bond carriers include proteins such as albumins, peptides, and polysaccharides such as aminodextran, each of which have multiple sites for the attachment of moieties. The nature of the carrier can be either soluble or insoluble for purposes of the invention.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Formulations to be used for in vivo administration must be sterile. This is readily accomplished by filtration through sterile filtration membranes.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Compositions can be prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249:1527, 1990 and Hanes, Advanced Drug Delivery Reviews 28:97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Toxicity of the active agents can be determined by standard pharmaceutical procedures in cell cultures or experimental animals, e.g., by determining the LD50 (the dose lethal to 50% of the population) or the LD100 (the dose lethal to 100% of the population). The dose ratio between toxic and therapeutic effect is the therapeutic index. The data obtained from these cell culture assays and animal studies can be used in further optimizing and/or defining a therapeutic dosage range and/or a sub-therapeutic dosage range (e.g., for use in humans). The exact formulation, route of administration and dosage can be chosen by the individual physician in view of the patient's condition.

“In combination with”, “combination therapy” and “combination products” refer, in certain embodiments, to the concurrent administration to a patient of the agents described herein in combination with additional therapies, e.g. physical therapy, surgery, and the like. When administered in combination, each component can be administered at the same time or sequentially in any order at different points in time. Thus, each component can be administered separately but sufficiently closely in time so as to provide the desired therapeutic effect.

“Concomitant administration” means administration of one or more components at such time that the combination will have a therapeutic effect. Such concomitant administration may involve concurrent (i.e. at the same time), prior, or subsequent administration of components. A person of ordinary skill in the art would have no difficulty determining the appropriate timing, sequence and dosages of administration.

The use of the term “in combination” does not restrict the order in which prophylactic and/or therapeutic agents are administered to a subject with a disorder. For example standard treatment at the time of stroke, e.g. anti-thrombolytic agents, anti-inflammatory agents, etc. are administered prior to the administration of an STC2 to a subject.

An individual being treated may be assessed for functional deficits at the ischemic episode is diagnosed, and can be performed in the period of recovery following the episode. The efficacy of treatment by the methods disclosed herein can be evaluated periodically, where the treatment may increase reduction of functional deficits by at least about 10%, at least about 20%, at least about 30%, at least about 40%, at least about 50%, at least about 60%, at least about 70%, at least about 80%, at least about 90%, at least about 100%, relative to standard of care or no treatment. The level of improvement can be determined, for example, in a clinical trial format. The assessment may use a stroke scale, as known in the art, imaging techniques, and the like as known in the art.

Stroke scales have been developed to standardize neurological evaluation in clinical trials. The best predictors of long-term outcome are believed to be level of consciousness, motor strength, gaze paresis, visual field deficits, and some aspects of postural control and language.

Stroke assessment scales aims to evaluate current cognitive and physical impairment. Stroke outcome scales, such as the Barthel index, are designed to determine the impact of strokes on daily activities and quality of life during stroke rehabilitation. The stroke assessment scale should also facilitate identification of new neurological deficits. For a review, see Sun et al. (2016) Br. J. Anasthesia 116 (3): 328-338; and Lyden and Hantson (1998) J. Stroke and Cerebrovascular Disease 7 (2): 113-127. These include the Barthel Index (Mahoney and Barthel, Maryland State Medical Journal, 14:56-61 (1965)), the Modified Rankin Scale (Rankin, Scot. Med., J. 2:200-215 (1957); van Swieten et al., Stroke, 19:604-607 (1988); Duncan et al., Stroke, 31:1429-1438 (2000)), the Glasgow Outcome Scale (Jennett and Bond, Lancet, 1 (7905): 480-4 (1975); Teasdale, J. Neuro. Neurosurg. Psychiatry, 41:603-610 (1978); Jennett et al., Lancet, 1:480-484 (1995)), and the National Institute of Health Stroke Scale (NIHSS) (Brott et al., Stroke, 20:864-870 (1989)). The methods of the present invention are suitable for the treatment of acute ischemic stroke of all stages of severity.

For example, the National Institutes of Health stroke scale (NIHSS) contains from 11 to 15 test items to measure acute stroke-related neurological deficit. It assesses level of consciousness, gaze, hemianopia, facial palsy, arm and leg strength, limb ataxia, sensory loss, neglect, dysarthria, and aphasia. The scores range from 0 (no deficit) to a maximum of 42. NIHSS can be used by both neurologists and nonspecialists with good agreement among observers. NIHSS test scores have been correlated with infarct volumes in brain imaging studies (r=0.5-0.8), thus confirming its construct validity.

Functional outcome scales are used to assess stroke related disability. They also may offer a functional prognosis, which is useful in evaluating efficacy, guiding rehabilitation programs, making placement decisions, and providing continuing care services for stroke survivors. Many functional outcome scales measure the patient's ability to perform ADLs.

The Barthel Index, (BI) is an ADL scale and frequently used measure of ADL competence in clinical stroke trials. It is also widely used to assess stroke-related disability outside the context of clinical trials. The BI consists of 10 items assessing feeding, chair/bed transfer, grooming, toileting, bathing, ambulation, stair climbing, dressing, bowel control, and bladder control. A score of 100 indicates full competence in all areas. Better scores are associated with independent living, shorter hospital stays and less need for intensive post-discharge services. Generally, a score greater than 60 indicates functional independence. The BI has been exhaustively studied, and concurrent validation has been reported by many groups. It has substantial interrater reliability (K=0.80) and excellent internal consistency (˜x=0.96). It has shown substantial agreement with the Katz Index of ADL and the Kenny Self-Care Evaluation.

Global outcome scales are a measure of handicap and a means of ranking patients into one of a limited number of broad categories. The most frequently used global outcome scales are the GOS and the Rankin Scale. The Rankin Scale is used to assess the extent of handicap after a stroke. It groups patients by ability to perform various activities and need for assistance, the modified form has 0- to 5-point scale, with 0 indicating no symptoms and 5 indicating severe disability.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Administration of Stanniocalcin 2 Post-Stroke Confers Functional Benefits

Stroke is a leading cause of long-term disability worldwide, intensifying the need for effective recovery therapies. Increased endogenous stem cell production corresponds with improved function. Transcriptome analysis identified stanniocalcin 2 (STC2) as one of the genes most significantly upregulated by electrical stimulation. Lentiviral upregulation and downregulation of STC2 in transplanted stem cells demonstrate that this glycoprotein is an essential mediator in the functional improvements seen with electrical modulation. Intraventricular administration of recombinant STC2 post-stroke confers functional benefits. Details may be found in Oh, B., Santhanam, S., Azadian, M. et al. Electrical modulation of transplanted stem cells improves functional recovery in a rodent model of stroke. Nat Commun 13, 1366 (2022).

### Introduction

Of the over 10 million strokes that occur annually, the majority of survivors are left with functional deficits. Early in development the brain is quite plastic, but as the human brain ages, its intrinsic capabilities to recover from insults such as stroke decline. Exogenous cell therapeutics including neural progenitor cell (NPC) delivery have emerged in an attempt to offset stroke-induced neuronal loss and promote functional recovery. Alternatively, augmentation of the brain's intrinsic mechanisms of repair, such as endogenous stem cell production, has been utilized to enhance restoration of function following damage from ischemia.

### Results

Combined electrical stimulation and NPC transplants upregulate endogenous stem cells. The neural environment created through electrical stimulation in tandem with cues generated from the transplanted NPCs enhanced the brain's intrinsic repair mechanisms, such as endogenous stem cell production. The production of endogenous stem cells in the subventricular zone (SVZ) increases after stroke. These endogenous stem cells travel to the ischemic area and impact recovery. An increase in production correlates to improved recovery, and manipulations to prevent endogenous stem cell production worsens stroke size and functional outcomes. Endogenous stem cells in the peri-infarct region are neural progenitors. Given prior work demonstrating endogenous stem cells enhance stroke recovery, the upregulation of endogenous stem cell production highlights an important potential mechanism to improve stroke recovery.

Electrical stimulation modifies NPC transcriptome. Electrical stimulation alters the properties of stem cells. Stanniocalcin-2 (STC2) was identified as an NPC gene that was one of the most significantly modified with electrical stimulation. Gene set enrichment analysis (GSEA) showed that 4 of the top 15 hallmark pathways have STC2 as the leading-edge gene. We verified the upregulation of STC2 using qRT-PCR and ELISA analysis. We further explored STC2 as a candidate in stroke recovery and the increase in endogenous stem cell production seen with combined stem cell delivery and electrical stimulation.

Upregulation of STC2 in NPCs improves functional stroke recovery and effects endogenous stem cell production. To substantiate STC2 in stem cell-enhanced recovery, we utilized lentiviral constructs to knock down (STC2^{KD}) or over-express STC2 (STC2^{UP}) in the NPCs delivered on a conductive polymer system. Scrambled constructs (Scramble^{KD}) served as controls. We determined that increased STC2 through electrical stimulation or lentiviral constructs (NPC^{Stim}, Scramble^{KD+Stim}, and STC2^{UP} respectively) improves functional recovery. If STC2 levels are reduced via lentiviral knockdown, the effect of electrical stimulation is lost, and recovery is similar to levels observed in Sham animals (STC2^{KD} and STC2^{KD+Stim}). With reduced NPC production of STC2, the benefit of the therapy in stimulated and unstimulated groups was diminished, demonstrating that STC2 is an important pathway for stem cell effects on stroke recovery, irrespective of its role in the improvement seen with electrical stimulation.

To determine if manipulation of the STC2 produced by the NPCs had similar effects on endogenous stem cell production, BrdU staining was performed. We found that only in the upregulated STC2 group and the scrambled electrically stimulated group (STC2^{UP} and Scramble^{KD+Stim} respectively) was an increase in endogenous NPCs with more mature mitotic markers (PAX6 and Nestin) observed. No change in stroke size was seen in any of the groups. Thus, STC2 is in an essential pathway for the increased production of endogenous stem cells in addition to functional recovery following stroke.

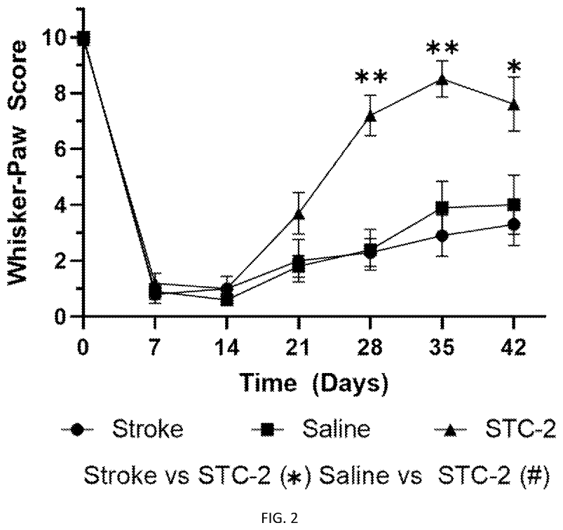

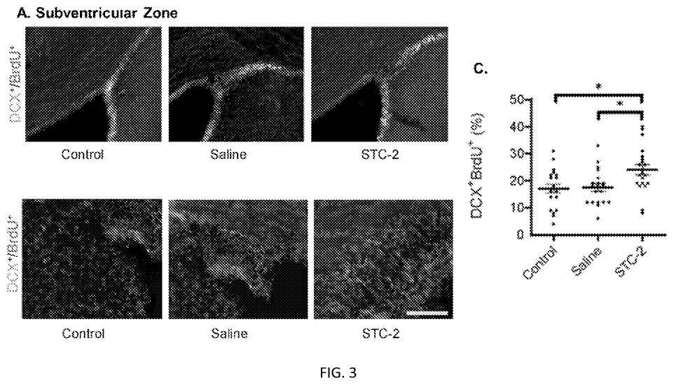

Intraventricular administration of STC2 improves functional stroke recovery. To further investigate the effect of STC2 alone on stroke recovery, we intraventricularly administered recombinant STC2 one-week post-stroke. Intraventricular administration facilitates circulation to various regions of brain such as SVZ and peri-infarct area via cerebrospinal fluid. We found that administration of STC2 leads to an improvement in functional recovery (FIG. 2) and to an increase in endogenous neuroblasts migrating toward the peri-infarct region (BrdU, with Doublecortin at 6 weeks post-stroke, FIG. 3). No change in the quantity of less mature mitotic markers (BrdU, with PAX6 and Nestin) were observed at the 6-week timepoint.

In vivo electrical stimulation of transplanted neural stem cells alters the repair mechanisms after stroke. See US2020/0261726 for details. We found that electrical stimulation alters the transcriptome of NPCs, and that STC2 is a critical upregulated molecule. STC2 is a secreted, homodimeric glycoprotein that is expressed in a wide variety of tissues including neurons with autocrine or paracrine functions. STC2 is primarily known for its role in cell turnover, specifically in tumors, and has been demonstrated to confer neuroprotective effects. Lentiviral transduction of NPCs with STC2 gain and loss of function demonstrates that STC2 is a critical mediator for enhanced functional recovery after stroke. Moreover, direct administration of recombinant STC2 leads to an increase in migratory neuroblasts toward the penumbral region and an improvement in behavioral outcome in our rodent stroke model.

### Methods

For STC2 gain and loss of function, hNPCs were treated with STC2 lentiviral activation particles (sc-405292-LAC) and STC2 shRNA (h) particles (sc-44127-V) respectively as per the manufacturer protocol (Santa Cruz Biotechnology Inc., Lentiviral activation particles transduction). Control shRNA lentiviral particles (sc-108080) were the control and referred to as Scramble^{KD} in this work. The Scramble^{KD} and STC2^{KD} groups were electrically stimulated as described in the above paragraph.

Cell viability assessments. In vitro immunostaining was performed on Day 3 (one day after electrical stimulation applied). Cell survival was determined by a Live/Dead kit (Life Technologies). The samples were incubated with 2 μL/mL of ethidium homodimer-1 and calcein AM for about 15 mins at 37° C. in the dark. After incubation, the cells were rinsed with 1×PBS, and imaged using a fluorescent microscope (Keyence BZ-X700), Four random, representative 0.34 mm×0.45 mm areas were analyzed, and alive and dead cells on the conductive scaffold were counted by a blinded individual with results averaged across the four areas (live cells/total cells).

Alternatively, cell viability testing using Alamar Blue has been applied to see the impact of electrical stimulation on the cells' survival. Briefly, 10% Alamar Blue cell viability reagent (Thermo Fisher Scientific) was added to each sample and incubated at 37° C. for 3 hours in the dark. The experimental groups were hNPCs with or without stimulation and control. After 3 hours of incubation, the absorbance of about 100 μL per sample was measured in duplicates at 570 and 600 nm using a multi-plate reader (SpectraMax, Molecular Devices). The percentage reduction in absorbance (percentage viability) was calculated with respect to total cells as per the manufacturer protocol.

RNA-Seq. In vitro electrically stimulated and unstimulated hNPC cDNA was isolated 24 hours following electrical stimulation as described in the “in vitro hNPC electrical stimulation” section (n=4 per group). RNA was extracted with the RNeasy Mini Plus kit (Qiagen, Hilden, Germany) after homogenization in Trizol (Life Technologies). cDNA was then synthesized using iScript cDNA Synthesis Kit (Bio-Rad, Hercules, CA), and purity was verified by the Agilent BioAnalyzer system.

Samples were prepped with Illumina TruSeq Stranded mRNA kit as per manufacturer's suggestion and sequenced as reverse paired-end on the HiSeq-4000 sequencer conducted at the Stanford SFGF as described previously. Raw fastq files was trimmed with Trimmomatic/0.36 and reads were aligned to the hg38 reference genome with STAR/2.5.1b aligner. Gene level counts were determined with STAR-quantMode option using gene annotations from GENCODE (p5). QC assessments such as unique alignment counts, unique/multiple ratio or exon/intron ratio was derived with ngsutilsj-0.3-2180ca6 using the bam-stats option. Differential gene expression and all other pathway analysis are conducted with R/3.5.3. Samples were imported, normalized with trimmed mean of M-values (TMM) from the EdgeR/3.24.3 package and further transformed with VOOM from the Limma/3.38.3 package. A linear model using the empirical Bayes analysis pipeline also from limma was then used to obtain p-values, adjusted p-values and log-fold changes (LogFC). Differential expressed genes (DEG) are defined with cutoffs FDR<0.05; p.value<0.05 and logFC>abs (1.5).

Gene Set Enrichment Analysis (GSEA) was performed on preranked logFC using the R package fgsea/1.13.5. Predefined pathways such as Hallmark gene sets and KEGG pathways were downloaded directly from the Molecular Signatures Database (MSigDB). Upon review of unsupervised clustering data using Tukey's outlier method, one of the unstimulated samples deviated remarkably from the other samples in the same experimental cohort and was removed from further analysis.

| Resource/software | Source |
|---|---|
| Hg38 human index and gtf | GENCODE |
| STAR aligner | |
| Trimmomatic/0.36 | |
| ngsutilsj-0.3-2180ca6 | |
| EdgeR | Bioconductor |
| Limma | Bioconductor |
| fgsea | Bioconductor |
| MSigDB | Broad Institute |

RNA extraction and qPCR. Total RNA was extracted from cells using a Qiagen RNeasy Plus Micro Kit (Qiagen). After accomplishing first-strand cDNA synthesis by iScript cDNA Synthesis Kit (Bio-Rad, Hercules, CA), quantitative real-time polymerase chain reaction (qRT-PCR) was performed with Taqman-polymerase and primers (Qiagen) for gene expression analysis. qRT-PCR was carried out on a QuantStduio 6 Flex Real-Time PCR System (ThermoFisher, Waltham, MA). The Delta-Delta CT method was utilized for relative expression levels with GAPDH as a housekeeping gene Taq polymerase and Taqman primers (Life Technologies) for GAPDH (Hs), STC2 (Hs01063215_m1), SNCB (Hs00608185_m1), NRN1 (Hs00213192_m1), TNNT1 (Hs00162848_m1), PLOD2 (Hs01118190_m1), PDEC4C (Hs00971865_m1), PPF1A4 (Hs00949811_m1), TMEM45A (Hs01046616_m1), and FGF11 (Hs00182803_m1) formed the qPCR reaction mixtures. The Delta-Delta CT method was utilized for qPCR analysis with the TUBB housekeeping gene and hNPCs grown on a glass chamber slide for 3 days as references.

ELISA STC2 and In-cell western blot analysis. Supernatant was collected from the hNPCs in vitro, on Day 3 after plating. A human STC2 ELISA kit (AB222880, Abcam) was used to assess STC2 concentrations according to manufacturer instructions. Samples were performed in duplicate with n=4 for each group.

For the in-cell western assay, the subcellular level of STC2 was quantified in situ using infrared (IR) intensity. After the electrical stimulation of STC2, the cells were plated in a 96-well plate (20,000 cells/well) and were immunolabeled with an IR-conjugated secondary antibody (IRDye® Secondary Antibodies, LiCor) using the standard immunocytofluorescence protocol. After the completion of the staining procedure, the plate was imaged using an Odyssey Fc IR imaging system (LiCor, Lincoln, NE). STC2 intensity was normalized to GAPDH expression by using the Odyssey CLx Imaging Studio 3.1 Analysis software.

dMCA occlusion and cell implantation. All animal procedures were approved by Stanford University's Administrative Panel on Laboratory Animal Care. Adult, male T-cell deficient nude rats (NIH-RNU 230±30 g) underwent distal middle cerebral artery (dMCA) occlusion model with occlusion of both common carotid arteries lasting 30 min as described previously. Rats were anesthetized with isoflurane with buprenorphine administered subcutaneously for analgesia. Ampicillin was in cage water 1 day prior to surgery (1 mg/ml) and for 7 days after transplantation.

One week after stroke, animals were randomized by vibrissae-whisker paw score, and implantation surgeries performed by a blinded individual. A craniectomy was drilled above the left cortical region between the neuroanatomical lambda and bregma markers, and the dural layer was excised. The conductive polymer system (cultured with hNPC cells for 24 hours; without any in vitro electrical stimulation) was removed from the in vitro system and implanted over the exposed brain tissue primarily on the penumbral cortex medial to the lesion after a phosphate buffered saline (PBS) wash (with approximately 5×10⁴ cells in hNPC groups or media alone for other groups). Surgicel (Ethicon) was placed over the implant to prevent movement with skin closure. A reference electrode attached to the cannula system was placed on the contralateral skull. The main cannula was secured to the contralateral skull with dental cement. Sham groups included animals with the dura opened but no implant.

For STC2 intraventricular delivery experiments, adult Sprague Dawley rats (300±30 g) underwent dMCA occlusion as described in the above paragraph, followed by STC2 osmotic pump implantation. About 24 hrs. prior to implantation, recombinant human STC2 protein (4 ng/ml of 1×PBS, Novus Biologicals) was loaded on to the osmotic pump (Azlet mini-osmotic pump model 2001, Braintree Scientific) and incubated at 37° C. as per the manufacturer protocol. The cannula from the osmotic pump was implanted at 0.8 mm posterior, 1.5 mm contralateral and 3.5 mm depth with respect to bregma. The reservoir was stored under the skin near the neck (back side). Control groups included a Sham (that underwent stroke, no treatment), and Saline (injected with 1×PBS without any STC2 protein in the osmotic pump).

In vivo hNPC electrical stimulation. Electrical stimulation (±800 mV, 100 Hz for 1 hr.) was applied to the rats receiving electrical stimulation for the first 3 days after implantation (implantation on Day 8, electrical stimulation on Day 9, Day10, and Day 11 post-implantation). The potential was applied across the polymer and the reference electrode while the animals were anesthetized as described above using a function generator (E3641A, Agilent).

Stroke volume and slice immunohistochemistry. At 3- or 6-week post-stroke, rats were perfused 4% paraformaldehyde (Sigma Aldrich) and 40 μm coronal slices were sectioned as described previously. Animals perfused at the 6-week time point were used for stroke volume analysis. Animals perfused at 3 weeks post-stroke were used for BrdU and immunostaining analysis.

BrdU intraperitoneal injections were performed 24 hours after the implantation of the conductive polymer system (post-stroke day 9). Briefly, BrdU was diluted in PBS to make a sterile solution of 10 mg/mL. A concentration of 100 mg/kg was intraperitoneally injected. Animals were sacrificed and fixed at 3 weeks post-stroke. Primary antibodies were incubated in blocking buffer at 4° C. overnight, followed by three 15-min PBS washes and detected by secondary antibodies (Alexa Fluor 488, 555 or 647, Thermofisher Scientific) as described previously 68. Samples were counter-stained with DAPI (Sigma-Aldrich) to visualize nuclei and mounted with Fluoromount Aqueous Medium (Sigma-Aldrich) before imaging. Samples were imaged on a Keyence All-in-One Fluorescence Microscope (BZ-X700, Keyence) using 20× or 60× objectives. Primary antibodies include anti-BrdU (1:200, Abcam), anti-GFAP (1:1000, Millipore Sigma), anti-Pax6 (1:100, Fisher Scientific), anti-NeuN (1:500, Cell Signaling Technology), anti-Nestin (1:200, Millipore Sigma), anti-PECAM (1:100, Millipore Sigma), anti-TUJ1 (1:100, Neuromics). Secondary antibodies were added as noted above. Images were analyzed on a Keyence optical microscope.

To assess for BrdU stained cells in the rat tissue, serial slices were taken 400 μm apart from the genu of the corpus collosum to the splenium. Cell counting were performed by a blinded observer. Peri-infarct area was defined as the area immediately surrounding the infarcted tissue and sampled in a 750 μm×650 μm window. The SVZ region was defined in the same manner as previously, briefly tissue next to the ventricles with higher cellular density was assessed and 750 μm×650 μm frame was obtained using a Keyence All-in-One Fluorescence Microscope (BZ-X700, Keyence) using 20× objectives. The midpoint between the peri-infarct region and SVZ region was then defined that as the middle area and assessed in the same manner. The threshold was then selected on one representative image and the same parameters were set for all images for counting purposes. Background signal was ignored, and the cells representative of true signal were then stereologically counted and averaged across slices for a given animal with n=4 per experimental group.

Stroke volume was assessed using cresyl violet staining 5 weeks after stroke as described previously. Serial slices were taken 400 μm apart from the genu of the corpus collosum to the splenium. Areas were calculated using the following equation

$$\frac{{Area}_{Contralateral} - {Area}_{Ipsilateral}}{{Area}_{Contralateral}} \times 100.$$

Assessments were performed by a blinded individual.

For quantification of TUJ1 (1:100, Neuromics) and GFAP (1:00, Invitrogen), four representative peri-infarct areas were selected from each ipsilateral slice at 400 μm intervals. These were selected at the same point in each slice by a blinded individual. ImageJ software was used by a blinded individual to calculate average fluorescence intensity against Tuj1 and GFAP.

Behavior analysis. Animals were divided into matched groups based on pre-transplant behavior testing (n=10 per group) and behavior testing was performed by blinded individuals. Functional recovery was assessed in two ways: 1) Using the modified neurologic severity scale (NSS) (Supplementary Table 4, a composite score assessing motor and sensory function including normal walking, walking on a beam, tactile and visual response when paw muscles are stimulated by touch) and 2) vibrissae-forepaw test. Animals were trained on 3 separate days prior to recording their baseline behavior. After baseline, the animals underwent dMCA occlusion and were tested 1 week after stroke prior to implantation. For the STC2 experiments, two animals died during the follow up and were removed from analysis (1 in the Sham and one in the STC2^{KD}). Animals without a significant deficit (significant deficit=vibrissae-forepaw score prior to implantation at <30% of baseline) were removed. The vibrissae-forepaw behavioral assay was evaluated for the potential confound of testing effect (by repeated measurements) via longitudinal assessments of the limb ipsilateral to the stroke as a non-effected control (Supplementary FIG. 22). The results do not suggest the presence of a testing effect however we acknowledge the limitation of this control being performed in stroked animals.

Statistical Analysis. Data was tested for normality and standard deviations using a normal quantile plot to determine the appropriate test (parametric vs non-parametric). Statistical evaluation was performed in GraphPad Prism or with genomic software, and one-way ANOVA or a Kruskal-Wallis test followed by Mann-Whitney or student t-test was utilized to determine significance at P<0.05. All tests were two-tailed unless performed as a confirmatory test. For RNA-Seq pathway analysis, a false discovery analysis was used to correct for multiple variables. Data are presented as mean±SEM.

### Example 2

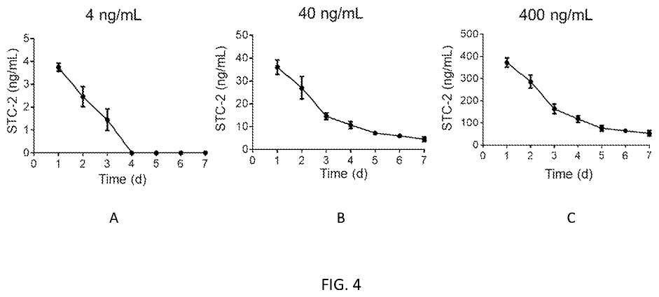

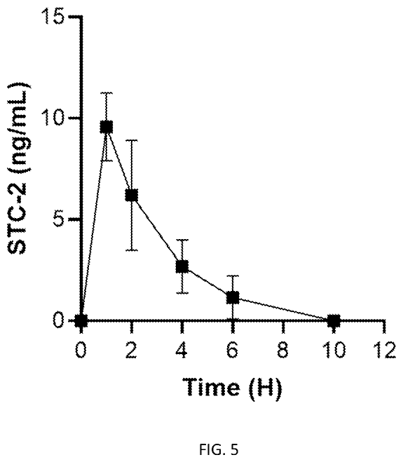

The stability of recombinant STC-2 was determined in vitro and in vivo. As shown in FIG. 4, the duration at which STC-2 would remain detectable with a standard STC-2 ELISA assay under normal physiological conditions (static artificial CSF at 37 degrees celsius) was determined, where the protein could be detected for longer than one week at concentrations of greater than 40 ng/ml. In vivo stability was tested by injection of STC-2 at a concentration of 10 mg/mL into the circulating CSF space via cisterna magna, and the protein was determined to be detectable up to about 10 h post-injection after a single bolus injection, shown in FIG. 5

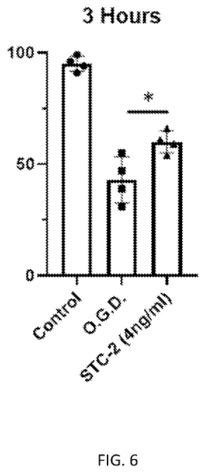

The neuroprotective dose for STC-2 to exhibit an effect on the viability of iPSC derived human neuronal cells (in culture) after oxygen and glucose deprivation. It was determined that 4 ng/ml of STC-2 conferred a significant neuroprotective effect on the neuronal cells, shown in FIG. 6.

### REFERENCES

1. Benjamin, E. J., et al. Heart Disease and Stroke Statistics-2017 Update: A Report From the American Heart Association. Circulation 135, e146-e603 (2017).
2. Sur, M., Garraghty, P. E. & Roe, A. W. Experimentally induced visual projections into auditory thalamus and cortex. Science (New York, N.Y.) 242, 1437-1441 (1988).
3. Knoflach, M., et al. Functional recovery after ischemic stroke—a matter of age: data from the Austrian Stroke Unit Registry. Neurology 78, 279-285 (2012).
4. Buhnemann, C., et al. Neuronal differentiation of transplanted embryonic stem cell-derived precursors in stroke lesions of adult rats. Brain: a journal of neurology 129, 3238-3248 (2006).
5. Daadi, M. M., Maag, A. L. & Steinberg, G. K. Adherent self-renewable human embryonic stem cell-derived neural stem cell line: functional engraftment in experimental stroke model. PloS one 3, e1644 (2008).
6. Guzman, R., et al. Intracarotid injection of fluorescence activated cell-sorted CD49d-positive neural stem cells improves targeted cell delivery and behavior after stroke in a mouse stroke model. Stroke; a journal of cerebral circulation 39, 1300-1306 (2008).
7. Stroemer, P., et al. The neural stem cell line CTX0E03 promotes behavioral recovery and endogenous neurogenesis after experimental stroke in a dose-dependent fashion. Neurorehabilitation and neural repair 23, 895-909 (2009).
8. Arvidsson, A., Collin, T., Kirik, D., Kokaia, Z. & Lindvall, O. Neuronal replacement from endogenous precursors in the adult brain after stroke. Nature medicine 8, 963-970 (2002).
9. Hao, X. Z., et al. Inhibition of Notch1 Signaling at the Subacute Stage of Stroke Promotes Endogenous Neurogenesis and Motor Recovery After Stroke. Frontiers in cellular neuroscience 12, 245 (2018).
10. Mine, Y., et al. Grafted human neural stem cells enhance several steps of endogenous neurogenesis and improve behavioral recovery after middle cerebral artery occlusion in rats. Neurobiology of disease 52, 191-203 (2013).
11. Parent, J. M., Vexler, Z. S., Gong, C., Derugin, N. & Ferriero, D. M. Rat forebrain neurogenesis and striatal neuron replacement after focal stroke. Annals of neurology 52, 802-813 (2002).
12. Shiromoto, T., et al. The Role of Endogenous Neurogenesis in Functional Recovery and Motor Map Reorganization Induced by Rehabilitative Therapy after Stroke in Rats. Journal of stroke and cerebrovascular diseases: the official journal of National Stroke Association 26, 260-272 (2017).
13. Hummel, F., et al. Effects of non-invasive cortical stimulation on skilled motor function in chronic stroke. Brain: a journal of neurology 128, 490-499 (2005).
14. Khedr, E. M., Ahmed, M. A., Fathy, N. & Rothwell, J. C. Therapeutic trial of repetitive transcranial magnetic stimulation after acute ischemic stroke. Neurology 65, 466-468 (2005).
15. Kleim, J. A., et al. Motor cortex stimulation enhances motor recovery and reduces peri-infarct dysfunction following ischemic insult. Neurological research 25, 789-793 (2003).
16. Murase, N., Duque, J., Mazzocchio, R. & Cohen, L. G. Influence of interhemispheric interactions on motor function in chronic stroke. Annals of neurology 55, 400-409 (2004).
17. Takeuchi, N., Chuma, T., Matsuo, Y., Watanabe, I. & Ikoma, K. Repetitive transcranial magnetic stimulation of contralesional primary motor cortex improves hand function after stroke. Stroke; a journal of cerebral circulation 36, 2681-2686 (2005).
18. Bible, E., et al. The support of neural stem cells transplanted into stroke-induced brain cavities by PLGA particles. Biomaterials 30, 2985-2994 (2009).
19. Bible, E., et al. Neo-vascularization of the stroke cavity by implantation of human neural stem cells on VEGF-releasing PLGA microparticles. Biomaterials 33, 7435-7446 (2012).
20. Nih, L. R., Gojgini, S., Carmichael, S. T. & Segura, T. Dual-function injectable angiogenic biomaterial for the repair of brain tissue following stroke. Nature materials 17, 642-651 (2018).
21. Schmidt, C. E., Shastri, V. R., Vacanti, J. P. & Langer, R. Stimulation of neurite outgrowth using an electrically conducting polymer. Proceedings of the National Academy of Sciences of the United States of America 94, 8948-8953 (1997).
22. George, P. M., et al. Electrical preconditioning of stem cells with a conductive polymer scaffold enhances stroke recovery. Biomaterials 142, 31-40 (2017).
23. George, P. M., et al. Engineered stem cell mimics to enhance stroke recovery. Biomaterials 178, 63-72 (2018).
24. Horie, N., et al. Transplanted stem cell-secreted vascular endothelial growth factor effects poststroke recovery, inflammation, and vascular repair. Stem cells (Dayton, Ohio) 29, 274-285 (2011).
25. George, P. M., et al. Fabrication and biocompatibility of polypyrrole implants suitable for neural prosthetics. Biomaterials 26, 3511-3519 (2005).
26. Encarnacion, A., et al. Long-term behavioral assessment of function in an experimental model for ischemic stroke. Journal of neuroscience methods 196, 247-257 (2011).
27. Garcia, J. H., Wagner, S., Liu, K. F. & Hu, X. J. Neurological deficit and extent of neuronal necrosis attributable to middle cerebral artery occlusion in rats. Statistical validation. Stroke; a journal of cerebral circulation 26, 627-634; discussion 635 (1995).
28. Zhang, R., et al. A nitric oxide donor induces neurogenesis and reduces functional deficits after stroke in rats. Annals of neurology 50, 602-611 (2001).
29. Li, Y. C., Tsai, L. K. & Young, T. H. Intraventricular infusion of a low fraction of serum enhances neurogenesis and improves recovery in a rodent stroke model. Neuroscience letters 611, 14-20 (2016).
30. Chang, K. A., et al. Biphasic electrical currents stimulation promotes both proliferation and differentiation of fetal neural stem cells. PloS one 6, e18738 (2011).
31. Song, S., et al. Controlling properties of human neural progenitor cells using 2D and 3D conductive polymer scaffolds. Scientific reports 9, 19565 (2019).
32. Stewart, E., et al. Electrical Stimulation Using Conductive Polymer Polypyrrole Promotes Differentiation of Human Neural Stem Cells: A Biocompatible Platform for Translational Neural Tissue Engineering. Tissue engineering. Part C, Methods (2014).
33. Subramanian, A., et al. Gene set enrichment analysis: a knowledge-based approach for interpreting genome-wide expression profiles. Proceedings of the National Academy of Sciences of the United States of America 102, 15545-15550 (2005). GAM: a web-service for integrated transcriptional and metabolic network analysis. Nucleic acids research 44, W194-200 (2016).
35. Byun, J. S., et al. Neuroprotective effects of stanniocalcin 2 following kainic acid-induced hippocampal degeneration in ICR mice. Peptides 31, 2094-2099 (2010).
36. Fazio, E. N., Dimattia, G. E., Chadi, S. A., Kernohan, K. D. & Pin, C. L. Stanniocalcin 2 alters PERK signalling and reduces cellular injury during cerulein induced pancreatitis in mice. BMC cell biology 12, 17 (2011).
37. Ito, D., et al. Characterization of stanniocalcin 2, a novel target of the mammalian unfolded protein response with cytoprotective properties. Molecular and cellular biology 24, 9456-9469 (2004).
38. Kim, P. H., Na, S. S., Lee, B., Kim, J. H. & Cho, J. Y. Stanniocalcin 2 enhances mesenchymal stem cell survival by suppressing oxidative stress. BMB reports 48, 702-707 (2015).
39. Law, A. Y. & Wong, C. K. Stanniocalcin-2 is a HIF-1 target gene that promotes cell proliferation in hypoxia. Experimental cell research 316, 466-476 (2010).
40. Huang, P., et al. Safety and Efficacy of Intraventricular Delivery of Bone Marrow-Derived Mesenchymal Stem Cells in Hemorrhagic Stroke Model. Scientific reports 9, 5674 (2019).
41. Reitmeir, R., et al. Vascular endothelial growth factor induces contralesional corticobulbar plasticity and functional neurological recovery in the ischemic brain. Acta neuropathologica 123, 273-284 (2012).
42. Jepsen, M. R., et al. The proteolytic activity of pregnancy-associated plasma protein-A is potentially regulated by stanniocalcin-1 and -2 during human ovarian follicle development. Human reproduction (Oxford, England) 31, 866-874 (2016).
43. Wu, F., et al. STC2 as a novel mediator for Mus81-dependent proliferation and survival in hepatocellular carcinoma. Cancer Lett 388, 177-186 (2017).
44. Chen, B., et al. STC2 promotes the epithelial-mesenchymal transition of colorectal cancer cells through AKT-ERK signaling pathways. Oncotarget 7, 71400-71416 (2016).
45. Yang, S., et al. STC2 promotes head and neck squamous cell carcinoma metastasis through modulating the PI3K/AKT/Snail signaling. Oncotarget 8, 5976-5991 (2017).
46. Yokobori, T., et al. Clinical significance of stanniocalcin 2 as a prognostic marker in gastric cancer. Annals of surgical oncology 17, 2601-2607 (2010).
47. Felling, R. J., et al. Neural stem/progenitor cells participate in the regenerative response to perinatal hypoxia/ischemia. The Journal of neuroscience: the official journal of the Society for Neuroscience 26, 4359-4369 (2006).
48. Jessberger, S. & Gage, F. H. Adult neurogenesis: bridging the gap between mice and humans. Trends in cell biology 24, 558-563 (2014).
49. Bergmann, O., et al. The age of olfactory bulb neurons in humans. Neuron 74, 634-639 (2012).
50. Jurkowski, M. P., et al. Beyond the Hippocampus and the SVZ: Adult Neurogenesis Throughout the Brain. Frontiers in cellular neuroscience 14, 576444 (2020).
51. Teramoto, T., Qiu, J., Plumier, J. C. & Moskowitz, M. A. EGF amplifies the replacement of parvalbumin-expressing striatal interneurons after ischemia. The Journal of clinical investigation 111, 1125-1132 (2003).
52. Adamczak, J., et al. Neurogenesis upregulation on the healthy hemisphere after stroke enhances compensation for age-dependent decrease of basal neurogenesis. Neurobiology of disease 99, 47-57 (2017).
53. Ahlenius, H., Visan, V., Kokaia, M., Lindvall, O. & Kokaia, Z. Neural stem and progenitor cells retain their potential for proliferation and differentiation into functional neurons despite lower number in aged brain. The Journal of neuroscience: the official journal of the Society for Neuroscience 29, 4408-4419 (2009).
54. Yamashita, T., et al. Subventricular zone-derived neuroblasts migrate and differentiate into mature neurons in the post-stroke adult striatum. The Journal of neuroscience: the official journal of the Society for Neuroscience 26, 6627-6636 (2006).
55. Oh, B., Levinson, A., Lam, V., Song, S. & George, P. Electrically Conductive Scaffold to Modulate and Deliver Stem Cells. Journal of visualized experiments: JoVE (2018).
56. Oh, B., et al. Modulating the Electrical and Mechanical Microenvironment to Guide Neuronal Stem Cell Differentiation. Advanced science (Weinheim, Baden-Wurttemberg, Germany) 8, 2002112 (2021).
57. Quail, M. A., et al. A large genome center's improvements to the Illumina sequencing system. Nature methods 5, 1005-1010 (2008).
58. Bolger, A. M., Lohse, M. & Usadel, B. Trimmomatic: a flexible trimmer for Illumina sequence data. Bioinformatics (Oxford, England) 30, 2114-2120 (2014).
59. Dobin, A., et al. STAR: ultrafast universal RNA-seq aligner. Bioinformatics (Oxford, England) 29, 15-21 (2013).
60. Harrow, J., et al. GENCODE: the reference human genome annotation for The ENCODE Project. Genome research 22, 1760-1774 (2012).
61. Breese, M. R. & Liu, Y. NGSUtils: a software suite for analyzing and manipulating next-generation sequencing datasets. Bioinformatics (Oxford, England) 29, 494-496 (2013).
62. Robinson, M. D., Mccarthy, D. J. & Smyth, G. K. edgeR: a Bioconductor package for differential expression analysis of digital gene expression data. Bioinformatics (Oxford, England) 26, 139-140 (2010).
63. Law, C. W., Chen, Y., Shi, W. & Smyth, G. K. voom: Precision weights unlock linear model analysis tools for RNA-seq read counts. Genome biology 15, R29 (2014).
64. Liberzon, A., et al. The Molecular Signatures Database (MSigDB) hallmark gene set collection. Cell systems 1, 417-425 (2015).
65. Liberzon, A., et al. Molecular signatures database (MSigDB) 3.0. Bioinformatics (Oxford, England) 27, 1739-1740 (2011).
66. Sergushichev, A. A. An algorithm for fast preranked gene set enrichment analysis using cumulative statistic calculation. bioRxiv, 060012 (2016).
67. Vos, J. G., Kreeftenberg, J. G., Kruijt, B. C., Kruizinga, W. & Steerenberg, P. The athymic nude rat. II. Immunological characteristics. Clinical immunology and immunopathology 15, 229-237 (1980).
68. Kelly, S., et al. Transplanted human fetal neural stem cells survive, migrate, and differentiate in ischemic rat cerebral cortex. Proceedings of the National Academy of Sciences of the United States of America 101, 11839-11844 (2004).
69. Ong, J., Plane, J. M., Parent, J. M. & Silverstein, F. S. Hypoxic-ischemic injury stimulates subventricular zone proliferation and neurogenesis in the neonatal rat. Pediatric research 58, 600-606 (2005).
70. Chen, J., et al. Intravenous administration of human umbilical cord blood reduces behavioral deficits after stroke in rats. Stroke; a journal of cerebral circulation 32, 2682-2688 (2001).
71. Schallert, T., Fleming, S. M., Leasure, J. L., Tillerson, J. L. & Bland, S. T. CNS plasticity and assessment of forelimb sensorimotor outcome in unilateral rat models of stroke, cortical ablation, parkinsonism and spinal cord injury. Neuropharmacology 39, 777-787 (2000).

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. A method of enhancing restoration of functional deficits following ischemia-induced damage to the brain of a subject, comprising:
administering to the subject an effective amount of a stanniocalcin 2 (STC2) agent.

2. The method of claim 1, wherein the STC2 agent is STC2 protein or a variant thereof.

3. The method of claim 2, wherein the STC2 protein is human STC2 protein.

4. The method of claim 1, wherein the STC2 agent is a polynucleotide encoding STC2 protein or a variant thereof.

5. The method of claim 4, wherein the STC2 protein is human STC2 protein.

6. The method of claim 1, wherein the STC2 agent is delivered to the subject by at least one of oral, parental or intra-CSF administration.

7. The method of claim 1, wherein the STC2 agent is delivered to the subject by intraventricular administration.

8. The method of claim 1, wherein a subject is selected for treatment following assessment that the individual has suffered ischemia-induced damage to the brain.

9. The method of claim 1, wherein administration of the STC2 agent is performed following ischemic damage.

10. The method of claim 1, wherein administration of the STC2 agent is performed after at least 1 day following ischemic damage.

11. The method of claim 1, wherein administration of the STC2 agent is performed after at least 3 days following ischemic damage.

12. The method of claim 1, wherein administration of the STC2 agent improves functional deficits in the subject.

13. The method of claim 1, wherein the ischemic damage is associated with an ischemic stroke, hemorrhagic stroke, or subarachnoid stroke.

14. A pharmaceutical composition for use in claim 1, comprising an STC2 agent and a pharmaceutically acceptable carrier.

15. The pharmaceutical composition of claim 14, formulated for intra-CSF delivery.
